(19) United States
(12) Patent Application Publication
(10) Pub. No.: US 2006/0121023 A1
(43) Pub. Date: Jun. 8, 2006

(54) Method to decrease nonspecific staining by Cy5

(76) Inventors: George Weiner, Iowa City, IA (US); Bernd Jahrsdoerfer, Iowa City, IA (US); Sue Blackwell, Iowa City, IA (US)

Correspondence Address: SCHWEGMAN, LUNDBERG, WOESSNER & KLUTH, 121 S. 8TH STREET, SUITE 1600, MINNEAPOLIS, MN, 55402, US

(21) Appl. No.: 11/005,886

(22) Filed: Dec. 7, 2004

(51) Int. Cl. A61K 39/395; A61K 48/00

(52) U.S. Cl. 424/133.1; 514/44

(57) ABSTRACT

Methods of reducing nonspecific binding of a fluorophore to cells expressing a Fc receptor, for example, CD64, is provided.

### STATEMENT OF GOVERNMENT RIGHTS

This invention was made, at least in part, with a grant from the Government of the United States of America (grant nos. CA77764 and CA97274 from the National Institutes of Health). The Government may have rights in the invention.

### BACKGROUND

Multiplex labeling of cells for analysis of mixed cell populations by flow cytometry employs fluorescence emission colors of known organic fluorophores in the visible-near-UV-near-IR spectral regions. Labeling with up to eight colors is currently possible. However, multiplex labeling of cells for flow cytometric analysis requires that the emission band of each distinct fluorophore not have substantial overlap with the emission band of other fluorophores employed in the analysis and that one or more of the fluorophore labeled molecules do not exhibit substantial nonspecific binding, which can increase background. For instance, van Vugt et al. (1996) reported that the indocarbocyanine (Cy5) portion of phycoerythrin-Cy5 (PE-Cy5) conjugated antibodies, which are widely used for multi-color flow cytometric analyses, bind to murine monocytes transfected with human CD64, the high affinity receptor for IgG (FcγRI). No such binding was seen with untransfected cells or cells transfected with other human Fc receptors (CD32, CD89) (Van Vugt et al., 1996). This non-Fab-specific binding has also been reported as nonspecific (i.e., independent of antibody specificity) staining of macrophages (Stewart and Stewart, 1993), and hampers the use of Cy5-containing immunoconjugates for flow cytometric analysis of human peripheral blood mononuclear cells (PBMC). Cells from patients treated with IFN-γ or G-CSF or from patients subject to acute inflammation upregulate immunoglobulin Fc receptors (FcR), and are therefore especially sensitive to this nonspecific Cy5 binding effect (Guyre et al., 1983; Repp et al., 1991; Davis et al., 1995). FcR blocking reagents including anti-FcR antibodies are expensive and inefficient in suppressing the binding of Cy5 conjugates to CD64.

What is needed is a simple way of effectively suppressing nonspecific binding of CyS conjugates to monocytes or other cells.

### SUMMARY OF THE INVENTION

The invention provides a method to reduce or eliminate binding of a Cy5 labeled ligand to cells expressing FcR such as a mixed cell population some of which likely express FcR. The method includes contacting a sample suspected of having cells comprising FcR, such as a physiological sample, a ligand comprising Cy5, and an effective amount of isolated nucleic acid, thereby yielding a mixture. In one embodiment, the sample is a blood sample, or cells isolated therefrom, such as isolated peripheral blood mononuclear cells, cultured cells or recombinant cells, i.e., those having exogenously introduced nucleic acid including those having deletions as a result of the exogenous introduction of the nucleic acid. In one embodiment, the sample is a peripheral blood, bone marrow or lymph node sample. For instance, the sample may be a peripheral blood sample from a patient treated with IFN-γ or G-CSF or suffering from acute inflammation. In one embodiment, the Cy5 labeled ligand is a Cy5 labeled antibody and in one embodiment a Cy5 and PE labeled antibody. The isolated nucleic acid may be DNA, RNA, chimeras thereof, single stranded or double stranded, linear or circular, and may include modified nucleotides, or any combination thereof. In one embodiment, the isolated nucleic acid is one or more distinct oligonucleotides, e.g., in one embodiment, the isolated nucleic acid is oligonucleotides each having the same length and sequence. In another embodiment, the oligonucleotides vary in sequence, vary in length, or both. In one embodiment, the method further comprises contacting the sample with one or more fluorophore labeled ligands labeled with a fluorophore other than Cy5, and optionally subjecting the resulting mixture to flow cytometry. Exemplary labels for use in combination with Cy5 in the invention include fluorescein (FITC), PE, PerCP, allophycocyanin (APC), Alexafluor488, Alexa647, Pacific Blue Alexafluor405, and Cy7, as well as those labels in combination with a different label such as PE, APC or PerCP. The fluorescence emissions may be measured simultaneously or sequentially, e.g., using flow cytometry.

As described herein, phosphorothioate oligodeoxynucleotides (PS-ODN) suppress the nonspecific binding of Cy5 labeled ligands in a sequence-independent manner. Binding of FITC-labeled PS-ODN to monocytes was blocked by CD64-specific monoclonal antibodies suggesting that CD64 is an oligonucleotide-binding protein. Thus, PS-ODN can be used as effective, simple and low-priced reagent to prevent nonspecific binding of Cy5 or PE-Cy5-conjugated antibodies to monocytes or other cells. The nonspecific fluorescence which is inhibited is that related to FcR and so is specific for FcR expressing cells, e.g., B cells, monocytes and macrophages, but not specific for any particular ligand labeled with Cy5. And as PE-Texas Red (PE-TR) shows moderate nonspecific fluorescence, but less than Cy5, the use of isolated nucleic acid with PE-TR labeled ligands may likewise reduce or eliminate nonspecific PE-TR binding to FcR bearing cells or nonspecific fluorescence by PE-TR labeled ligands.

Accordingly, the invention also provides a method to reduce or eliminate nonspecific fluorescence by Cy5 labeled antibodies in a sample comprising cells suspected of expressing FcR. The method includes contacting the sample with a Cy5 labeled antibody and an amount of isolated nucleic acid effective to reduce or eliminate nonspecific fluorescence by the Cy5 labeled antibodies, thereby yielding a mixture. Specific fluorescence may then be detected or determined, e.g., by flow cytometry.

Also provided are compositions and kits comprising Cy5 labeled ligands and isolated nucleic acid.

### BRIEF DESCRIPTION OF THE DRAWINGS

FIG. 1. Binding of PE-Cy5-conjugated anti-CD19 to monocytes in the presence of different agents. PBMC were isolated from healthy blood donors and stained with PE-Cy5-conjugated anti-CD19 monoclonal antibodies (mAB) and PE-conjugated anti-CD14 mAB in the presence of a phosphorothioate PS- or a phosphodiester (PO)-oligonucleotide (ODN) at 5 μg/ml, anti-CD64 mAB at 20 μg/ml (clone 10.1, Serotec Ltd., Oxford, UK), human IgG at 1 mg/ml (Sigma, St. Louis, Mo.) or heparin at 200 U/ml (Elkins-Sinn Inc, Cherry Hill, N.J.). Alternatively, anti-CD64 clones M22 or 32.2 were used instead of clone 10.1 with similar results. Cells were washed twice and analyzed by flow cytometry. Numbers in the figures indicate the percentage of PE-Cy5-positive monocytes based on all CD14-positive cells. Data are representative for 5 different experiments with similar results.

FIG. 2. Inhibition of PE-Cy5 binding to monocytes by ODN. PBMC were stained with PE-Cy5-conjugated anti-CD19 mAB and PE-conjugated anti-CD14 mAB in the presence of different concentrations of PS-ODN or PO-ODN. Experiments were repeated at least 4 times with different PE-Cy5-conjugated mAB and different ODN sequences, yielding similar results.

FIG. 3. Binding of PS-ODN to monocytes in the presence of anti-CD64 antibodies. PBMC were incubated in the presence of a FITC-labeled PS-ODN (see Table 1) and increasing concentrations of anti-CD64 mAB or control IgG at 37° C. for 3 hours. Cells were then harvested, washed, stained for CD14 and analyzed by flow cytometry. One representative experiment out of 3 with similar results is shown in experimental duplicates.

### DETAILED DESCRIPTION OF THE INVENTION

A “nucleic acid”, as used herein, is a covalently linked sequence of naturally occurring nucleotides or a sequence which includes modified nucleotides, including deoxyribonucleotides or ribonucleotides, in which the 3′ position of the pentose of one nucleotide is joined by a group, e.g., a phosphodiester group, to the 5′ position of the pentose of the next, and in which the nucleotide residues (bases) are linked in specific sequence, i.e., a linear order of nucleotides, including double- and single-stranded molecules. Nucleic acid may comprise modified nucleotides, such as methylated or capped nucleotides and modification of the sugar, base, and/or phosphate groups, and may be interrupted by non-nucleotide components. The sugar groups of the nucleotide subunits may be ribose, deoxyribose, or modified derivatives thereof such as 2′-O-methyl (“2′-O—Me”) ribose (2′ methoxy, “2′-MeO”, derivative of deoxyribose), or arabinose derivatives. The nucleotide subunits may be joined by linkages such as phosphodiester linkages, modified linkages or by non-nucleotide moieties. Modified linkages include those in which a standard phosphodiester linkage is replaced with a different linkage, such as a phosphorothioate, phosphoramidate, phosphorodithioate, phosphate triester, O-methylphosphoroamidite, methylphosphonate, or peptide nucleic acid linkage, and including morpholino modified oligo- or polynucleotides. Nitrogenous base analogs also may be components of nucleic acid in accordance with the invention. If present, modifications to the nucleotide structure may be imparted before or after assembly of a polynucleotide or an oligonucleotide. A “polynucleotide”, as used herein, is nucleic acid containing a sequence that is greater than about 250 nucleotides in length. An “oligonucleotide”, as used herein, is defined as a molecule comprised of 2 or more deoxyribonucleotides or ribonucleotides, preferably more than 3, and usually more than 10, but less than 250, preferably less than 200, deoxyribonucleotides or ribonucleotides. The nucleic acid may be generated in any manner, including chemical synthesis, DNA replication, amplification, e.g., polymerase chain reaction (PCR), reverse transcription (RT), or a combination thereof.

Linear nucleic acid molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus. For instance, DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring.

As used herein, the terms “isolated ” refers to in vitro preparation, isolation and/or purification of a nucleic acid or population of cells so that they are not associated with substances they are associated with in nature or free from at least one contaminating substance they are normally associated with in vivo. Thus, for example, an isolated substance may be prepared by using a purification technique to enrich it from a source mixture. The term “isolated” when used in relation to a nucleic acid, as in “isolated oligonucleotide” or “isolated polynucleotide” refers to a nucleic acid sequence that is present in a form or setting that is different from that in which it is found in nature, i.e., separated from at least one contaminant with which it is ordinarily associated in its source, e.g., an oligonucleotide is separated from dNTPs. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. Hence, with respect to an “isolated nucleic acid”, which includes an oligonucleotide, a polynucleotide of genomic, cDNA, or synthetic origin or some combination thereof, the “isolated nucleic acid” (1) is not associated with all or a portion of a polynucleotide in which the “isolated nucleic acid ” is found in nature, (2) is operably linked to a polynucleotide which it is not linked to in nature, or (3) does not occur in nature as part of a larger sequence. The isolated nucleic acid may be present in single-stranded or double-stranded form.

“Ligand” refers to a component which specifically binds to all or a portion of another molecule, such as a cell surface receptor, or intracellular molecule. In one embodiment, a ligand useful in this invention is an antibody or a functional fragment thereof capable of binding to a cell surface receptor on mononuclear cells. Such antibodies or fragments may be defined to include polyclonal antibodies from any native source, and native or recombinant monoclonal antibodies of classes IgG, IgM, IgA, IgD, and IgE, hybrid (chimeric) derivatives, and fragments of antibodies including Fab, Fab′ and F(ab′)2, humanized or human antibodies, recombinant or synthetic constructs containing the complementarity determining regions of an antibody, and the like. In one embodiment, a ligand of the invention is characterized by the desired ability to bind a specified cell surface receptor on a population of white blood cells.

As used herein, the term “antibody” refers to a protein having one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies may exist as intact immunoglobulins, or as modifications in a variety of forms including, for example, FabFc₂, Fab, Fv, Fd, (Fab′)₂, an Fv fragment containing only the light and heavy chain variable regions, a Fab or (Fab)′₂ fragment containing the variable regions and parts of the constant regions, a single-chain antibody, e.g., scFv, CDR-grafted antibodies and the like. The heavy and light chain of a Fv may be derived from the same antibody or different antibodies thereby producing a chimeric Fv region. The antibody may be of animal (especially mouse or rat) or human origin or may be chimeric or humanized. As used herein the term “antibody” includes these various forms.

A “sample” refers to a sample having or suspected of containing cells, which sample may be obtained from an organism, e.g., it can be a physiological sample, such as one from a human patient, a laboratory mammal such as a mouse, rat, pig, monkey or other member of the primate family, by drawing a blood sample, spinal fluid sample, bone marrow sample or urine sample, a needle aspirate from tissues, or a dissociated tissue sample, e.g., one obtained after mincing or otherwise dissociating solid tissue including tumor tissue, a culture of such a sample, cells from permanent cell lines, or may include recombinant cells, e.g., those altered by recombinant techniques, or any combination of cells. In one embodiment, the cells may be isolated cells, for instance, peripheral blood mononuclear cells isolated from blood.

With flow cytometry of properly prepared cells, using molecules which specifically bind to cell surface antigens, e.g., polyclonal or monoclonal antibodies, bind to intracellular molecules and are permeable to the cell membrane, or bind to intracellular molecules in permeabilized cells, it is possible to analyze various types of cells. The ability to differentiate and phenotype cells including blood cells is useful for evaluating disease states and other health conditions in living beings. One popular technique for cell differentiation and lymphocyte immunophenotyping is flow cytometry. With flow cytometry, cells from an appropriately prepared blood sample, are passed one at a time through a flow cell, which is adapted for sensing or detecting impedance changes, light scatter or some other characteristic of the cell. Some flow cytometry instruments are equipped with detectors for measuring emissions from fluorescent molecules (fluorophores) that may be associated with the cells, while other detectors measure scatter intensity or pulse duration. Data about cells that pass through the flow cell can be plotted on a cytogram according to the measured property.

During the flow cytometry process for nucleated cells in blood or other physiological samples, it may be desirable to eliminate the presence of erythrocytes (red blood cells) in the sample. Accordingly, during sample preparation, which-may be done by manual, semi-automated or automated techniques), a lytic reagent may be employed for lysing red blood cells and thereafter isolating the leukocyte (white blood cell) populations. Leukocytes are known to include a myeloid fraction of monocytes and granulocytes (neutrophils, basophils and eosinophils) and a lymphoid fraction (namely NK, B and T cell lymphocytes). Each of the populations may be distinguished based upon the distinctive cell surface antigens or markers. Moreover, within each category of lymphocytes, there are sub-categories, such as “helper” T cells or “suppressor” T cells, the latter of which also includes several subsets, distinguishable by their respective surface markers.

For instance, to prepare a sample for fluorescent flow cytometry, according to one conventional practice, a volume of fresh sample blood is provided, and a suitable amount one or more desired fluorophore labeled ligands such as fluorophore labeled monoclonal or polyclonal antibodies are added. Alternatively, a primary antibody (those without a fluorophore) is added, then a fluorophore labeled second antibody which binds the primary antibody is added. The primary and secondary antibodies may be polyclonal, monoclonal or chimeric antibodies, or any combination thereof. The sample and antibody mixture is incubated to allow antibody/antigen binding to take place. In one embodiment, after incubation, a lytic reagent may be added to lyse erythrocytes in the sample. The debris from the lysing of the erythrocytes is optionally removed, by washing, leaving a sample of leukocytes with antibodies bound to cells with the appropriate ligands. The sample is optionally fixed and run through a fluorescent detecting flow cytometry instrument or observed with a fluorescence microscope. The presence of a particular fluorophore on the cell's surface or internally would indicate the occurrence of a specific antigen-antibody reaction.

Individual dyes or fluorophores or tandem dyes may be used to label ligands. Tandem dyes are non-naturally occurring molecules which may be formed of a phycobiliprotein and another dye. See, for example, U.S. Pat. Nos. 4,542,104 and 5,272,257, incorporated by reference herein. Phycobiliproteins are a family of macromolecules found in red algae and blue-green algae. The biliproteins (the term “biliproteins” is equivalent to the term “phycobiliprotein”) have a molecular weight of at least about 30,000 daltons, more usually at least about 40,000 daltons, and may be as high as 60,000 or more daltons usually not exceeding about 300,000 daltons. The biliproteins normally are comprised of from 2 to 3 different subunits, where the subunits may range from about 10,000 to about 60,000 molecular weight. The biliproteins are normally employed as obtained in their natural form from a wide variety of algae and cyanobacteria. Examples of phycobiliproteins useful in the present invention are phycocyanin, allophycocyanin (APC), allophycocyanin B, phycoerythrin (PE) and R-phycoerythrin.

For instance, for multiple, e.g., three color, flow cytometry, fluorophores may be selected from a phycobiliprotein such as APC and PE, (e.g., B— or R-type), propidium iodide, Texas Red (TR), fluorescein isothiocyanate (FITC), peridinin chlorophyl protein (PerCP), Cy3, Cy5, or tandem dyes such as PE-Cy5, PE-Cy7, and PE-TR. Any of these can be used to label the primary and secondary antibodies of the invention using conjugation methods well known in the art.

FITC labeled ligands can generally be used with any flow cytometer equipped with an argon laser that emits 488 nm light. The peak emission of FITC is approximately 525 nm and may be detected in the FL-1 channel. FITC labeled ligands can also be used for fluorescence microscopy.

PE labeled ligands can generally be used with any flow cytometer equipped with a laser that emits 488 nm light. The peak emission of PE is approximately 575 nm and may be detected in the FL-2 channel.

Cy3 labeled ligands can be used with equipment having a laser that emits 488 nm light, and the peak emission of Cy3 is approximately 565 nm. CyS labeled ligands can be used with equipment that emits a laser at about 633 or 635 nm light. The peak emission of Cy5 is about 667 nm.

TR conjugates are useful in multi-color flow cytometry with instruments equipped with a second laser that excite TR within its absorbance range. TR can be used with fluorescent microscopes equipped with the proper filters.

APC labeled ligands are useful in multi-color flow cytometry with instruments equipped with a second laser (e.g., HeNe or red diode) that excite APC within its absorbance range.

Other markers which may be employed to provide additional colors are fluorescent proteins, e.g., green fluorescent protein, blue fluorescent protein, yellow fluorescent protein and red fluorescent protein; also useful may be markers which emit upon excitation by ultraviolet light.

Examples of tandem dyes useful in the present invention are PE-TR, PE-Cy5, PE-Cy7, APC-Cy5, and APC-Cy7. PE-Cy5 is excited at 488 nm by an argon laser. The emission of PE-Cy5 begins at approximately 650 nm and peaks at 667 run. When used on a Becton Dickson (BD) FACScan™ or FACSCalibur™ PE-Cy5 may be detected in the FL-3 channel. When used on a Coulter EPICS® XL it may be detected in the FL-4 channel. PE-Cy7 is excited at 488 nm by an argon laser. The emission of PE-Cy7 begins at approximately 700 nm and peaks at 776 nm. Its emission is detected at various channels depending upon the filter arrangement of the flow cytometer. When used on a BD FACScan™ or FACSCalibur™, it is detected in FL-3. On larger instruments such as the FACS Vantage™, PE-Cy7 is detected in FL-6. When used with FITC, PE and PE-Cy5 on a Coulter EPICS® XL certain filters must be changed.

PE-TR is excited at 488 nm by an argon laser. The emission of PE-TR peaks at 615 nm. When used on BD instruments, it is typically detected in the FL-3 channel. When used on a Coulter EPICS XL it is typically detected in the FL-3 channel as well.

APC-Cy7 is excited by a 633 or 635 nm emitting laser. The peak emission of APC-Cy7 is 776 nm.

The compositions of the present invention which may include a ligand, a labeled ligand, an immunostain, dye, a fluorophore or other label, and/or isolated nucleic acid, packaged separately or together, may be supplied as part of a kit, whose other components might include controls, other chemical reagents for a cytometry lab, a hematology instrument, a flow cytometer, sample preparation instruments, data management units or the like. For instance, isolated nucleic acid and a Cy5 labeled ligand can be packaged individually or together.

The compositions of the present invention are useful for performing hematology analysis of samples in manual and automated instruments, with flow cytometers with hematology blood analyzers, with microscopy using optical microscopy, electron microscopy or the like. Samples prepared for analysis with the compositions of the present invention may be prepared using manual, semiautomated or automated techniques.

To prepare a sample for fluorescent flow cytometry, according to one method of the present invention, a predetermined volume of fresh sample blood is provided, and a suitable amount of a desired fluorophore labeled antibody is added. The sample and antibody mixture is then incubated for a predetermined time (e.g., about 10 to about 30 minutes) at a predetermined temperature to allow antibody and antigen binding to take place. The sample may then be washed and resuspended as desired. A different fluorophore labeled antibody, which optionally recognizes a different ligand, may be added simultaneously with or sequentially to the first labeled antibody. After incubation, the composition of the present invention that is contacted with the sample may be contacted with a lysing agent to lyse erythrocytes in the sample. Alternatively, the sample may be one that is separated from erythrocytes or other contaminating cells, e.g., using density gradient separation. To eliminate erythrocytes in the sample, the sample is contacted with a lysing agent for a period of time sufficient so that any erythrocytes that remain in the sample are not materially distort measurements, but not so long that leukocytes are damaged. The debris from the lysing of the erythrocytes may optionally be removed, by washing, leaving a sample of leukocytes with antibodies bound to cells with complementary surface antigens. The sample is then run through a fluorescence detecting flow cytometry instrument. In another embodiment, the composition of the present invention is contacted with the cells prior to labeling and incubation.

In accordance with the above, it will be appreciated that among the advantages of the compositions of the present invention are that the compositions of the present invention can be formulated as nontoxic compositions. The compositions can be used in wash and no wash systems. Samples may be lysed before or after staining with labeled ligands, with no adverse effect upon fluorescence or disruption of cell surface markers. The compositions may be used in conjunction with a fixation-permeation agent. The compositions are suitable for use in a variety of commercially available instruments, such as (without limitation) available from Becton Dickinson under its FACS™ designation, such as (without limitation) FACSCalibur, FACSVantage, or instruments employing like technology; from Beckman Coulter under the designation EPICS® (as well as associated sample preparation stations such as its Q-PREP line; from Abbott Laboratories under the CELL-DYN™ designation (e.g., Cell-Dyn 4000).

The invention will be further described by the following non-limiting example.

### EXAMPLE

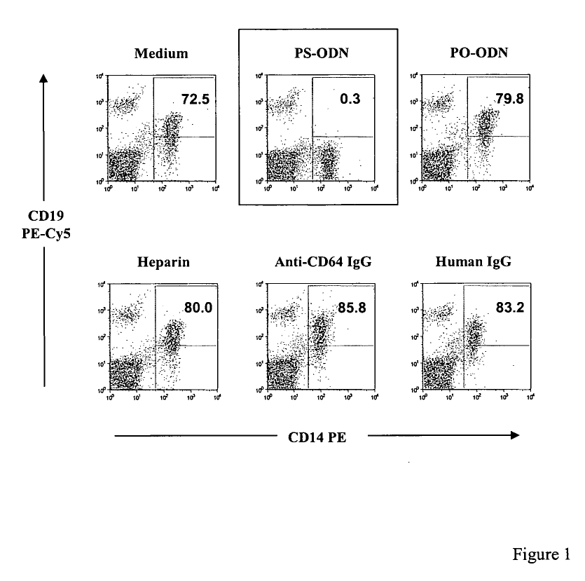

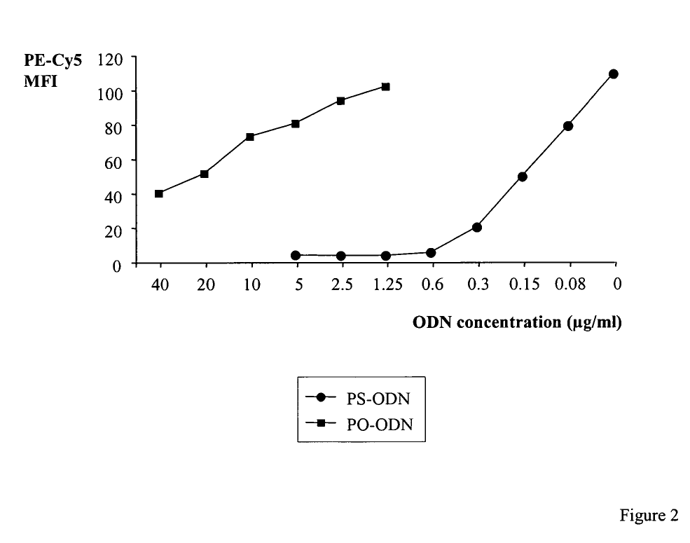

Staining with PE-Cy5- or Cy5-labeled antibodies against a variety of antigens showed nonspecific binding of these antibodies to monocytes. While exploring the effects of various PS-ODN on PBMC subsets, PS-ODN were found to be capable of blocking this nonspecific staining. Nonspecific staining was seen in untreated control samples, but not in samples treated with PS-ODN. This effect was not ODN sequence-specific, as it was seen with a variety of different ODN sequences including both immunostimulatory and non-immunostimulatory PS-ODN (Table 1). Kinetic experiments demonstrated that the blocking effect was very rapid, as there was no need for preincubation of PBMC with PS-ODN. Staining of PBMC with PE-Cy5-conjugated antibodies in the same solution as PS-ODN for 20 minutes on ice or at room temperature demonstrated complete blockage of nonspecific binding of PE-Cy5 conjugates to monocytes (FIG. 1). Similar results were seen with Cy5-conjugated antibodies. PS-ODN at 0.15 μg/ml resulted in half-maximal inhibition of nonspecific binding of the PE-Cy5-conjugated antibodies but did not block specific binding of the PE-Cy5- or Cy5-conjugated antibodies to their target antigen. Maximal inhibition of nonspecific binding was reached at PS-ODN concentrations as low as 0.6-1.25 μg/ml (FIG. 2). Similar results were obtained in monocytes cultured overnight with 1000 U/ml IFN-γ or 300 U/ml G-CSF (both from PeproTech Inc, Rocky Hill, N.J.) (data not shown).

TABLE 1
Sequences of ODN used in this study

| | |
|---|---|
| Phosphorothioate oligonucleotides | Phosphodiester oligonucleotides |

| | |
|---|---|
| 5′-tcg tcg ttt tgt cgt ttt gtc gtt-3′*, ** | 5′-tcg tcg ttt tgt cgt ttt gtc gtt-3′* |
| (SEQ ID NO:1) | (SEQ ID NO:7) |
| 5′-tzg tzg ttt tgt zgt ttt gtz gtt-3′*** | 5′-tag cac agc ctg gat agc aac gta-3′ |
| (SEQ ID NO:2) | (SEQ ID NO:8) |
| 5′-tgc tgc ttt tgt gct ttt gtg ctt-3′* | |
| (SEQ ID NO:3) | |
| 5′-ggg gga cga tcg tcg ggg gg-3′* | |
| (SEQ ID NO:4) | |
| 5′-ggg gga gca tgc tgg ggg gg-3′ | |
| (SEQ ID NO:5) | |
| 5′-tcg tcg ttt tcg gcg cgc gcc g-3′* | |
| (SEQ ID NO:6) | |

* immunostimulatory sequence
** also used FITC-conjugated at the 5′-end
*** z = 5-methyl cytosine

PS-ODN have a phosphorothioate backbone, which by itself can have modulating effects on monocytes and macrophages (Sester et al., 2000). Therefore, it was evaluated whether the presence of the PS-backbone was important for the observed effect. Phosphodiester ODN (PO-ODN) and PS-ODN were compared for their ability to block nonspecific binding of PE-Cy5 conjugates to monocytes. PO-OND had a modest effect on PE-Cy5 binding, but this effect was considerably weaker than that seen with PS-ODN, indicating the PS-backbone allows for an enhanced blocking effect (FIG. 2).

One surface molecule expressed on monocytes and known to efficiently bind polyanionic macromolecules like PS-ODN or heparin is Mac-1 (CD11b/CD18) (Benimetskaya et al., 1997). Since Mac-1 appears to be a signaling partner for a series of different receptors including Fc receptors (Petty and Todd, 1996), the involvement of Mac-1 in the effects described above for PS-ODN was tested by using heparin instead of PS-ODN. Heparin at concentrations of up to 200 U/ml did not inhibit PE-Cy5 binding to monocytes (FIG. 1), nor did increasing concentrations of heparin reverse the inhibiting effect of PS-ODN on PE-Cy5 binding to monocytes, suggesting that Mac-1 is not involved in the effects observed with PS-ODN.

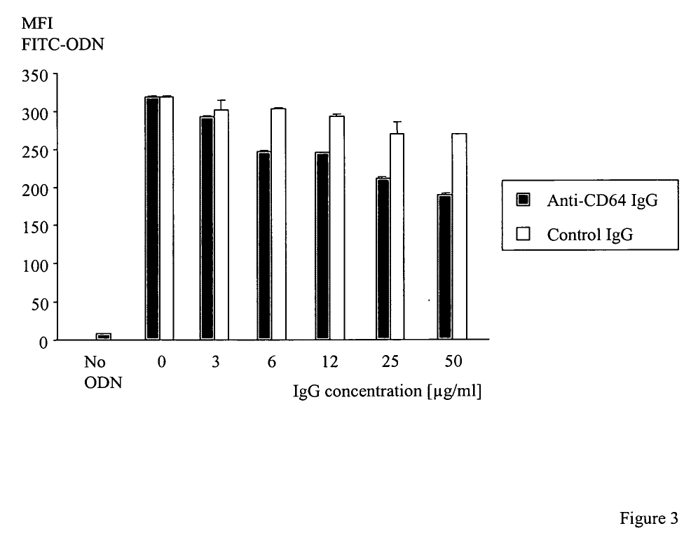

These results demonstrate that Cy5 binding is blocked by PS-ODN, and, when combined with those of van Vugt et al. demonstrating that Cy5 binds to CD64, suggest that CD64 may play a role in the ability of PS-ODN to inhibit the binding of Cy5 to monocytes. Therefore, it was evaluated whether the opposite was true, namely whether blocking of CD64 could inhibit PS-ODN binding to monocytes by using FITC-labeled PS-ODN. As expected, FITC-labeled PS-ODN (Integrated DNA Technology, Coralville, Iowa, USA; see Table 1 for sequence) bound to monocytes in PBMC. Preincubation of PBMC with murine anti-human CD64 mAB (clone 10.1), but not with murine control IgG (both from Serotec Ltd, Oxford, UK), partially blocked the binding of PS-ODN to monocytes in a concentration-dependent manner (FIG. 3). These data suggest that CD64 is involved in binding of PS-ODN to monocytes.

No blocking of PE-Cy5 binding to monocytes was found with either a commercially available Fc receptor blocking reagent (Miltenyi Biotec, Auburn, Calif.) or with various anti-CD64 antibodies (clone 10 from Serotec Ltd, Oxford, UK: FIG. 1; clones M22 and 32.2 from Abcam, Inc., Cambridge, Mass.) at up to 50 μg/ml. Taken together, these data suggest the epitope responsible for Cy5 binding is the same as that responsible for binding of PS-ODN and may include both CD64 and other monocyte antigens. The Cy5-binding epitope appears to be different from that targeted by Fc receptor blocking reagents and the different clones of anti-CD64 antibodies.

In conclusion, the studies outlined above demonstrate that PS-ODN can block the binding of Cy5 conjugates to CD64 on monocytes. These findings have potential immediate utility in that the addition of PS-ODN as blocking agents allows for the use of PE-Cy5 and CyS-conjugated mAB for flow cytometry, both in the laboratory and in the clinic. The data further suggest that CD64 may represent an oligonucleotide-binding molecule on monocytes. This could have significant implications for PS-ODN-based therapeutics.

### REFERENCES

Benimetskaya et al. (1997) Mac-1 (CD11b/CD18) is an oligodeoxynucleotide-binding protein. Nat. Med., 3, 414-20.
Davis et al. (1995) Neutrophil CD64 expression: Potential diagnostic indicator of acute inflammation and therapeutic monitor of interferon-g therapy. Lab.

Hematol., 3.
Guyre et al. (1983) Recombinant immune interferon increases immunoglobulin G Fc receptors on cultured human mononuclear phagocytes. J Clin. Invest., 72, 393-7.
Petty et al. (1996) Integrins as promiscuous signal transduction devices. Immunol Today, 17, 209-12.
Repp et al. (1991) Neutrophils express the high affinity receptor for IgG (Fc gamma RI, CD64) after in vivo application of recombinant human granulocyte colony-stimulating factor. Blood, 78, 885-9.
Sester et al. (2000) Phosphorothioate backbone modification modulates macrophage activation by CpG DNA. J. Immunol., 165, 4165-73.
Stewart et al. (1993) Immunological monitoring utilizing novel probes. Ann. NY Acad. Sci., 677, 94-112.
van Vugt et al. (1996) Binding of PE-CY5 conjugates to the human high-affinity receptor for IgG (CD64). Blood, 88, 2358-61.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to reduce or eliminate binding of a Cy5 labeled ligand to cells expressing a Fc receptor (FcR), comprising:
contacting a sample suspected of having cells comprising FcR, a ligand comprising Cy5, and an amount of isolated nucleic acid effective to inhibit or eliminate binding of Cy5 to FcR, thereby yielding a mixture.

2. The method of claim 1 wherein the sample comprises monocytes.

3. The method of claim 1 wherein the sample comprises mammalian peripheral blood mononuclear cells.

4. The method of claim 3 wherein the cells are isolated human peripheral blood mononuclear cells.

5. The method of claim 1 wherein the cells express recombinant FcR.

6. The method of claim 1 wherein the ligand comprising CyS is an antibody comprising CyS.

7. The method of claim 1 wherein the ligand comprising CyS is an antibody comprising PE and CyS.

8. The method of claim 6 or 7 wherein the antibody is a humanized antibody.

9. The method of claim 6 or 7 wherein the antibody is a monoclonal antibody.

10. The method of claim 1 wherein the isolated nucleic acid includes one or more oligonucleotides.

11. The method of claim 1 wherein the isolated nucleic has a phosphorothioate backbone.

12. The method of claim 1 wherein the isolated nucleic acid is isolated oligonucleotides each having the same sequence.

13. The method of claim 1 wherein the isolated nucleic acid include nucleotides with a modified sugar, a modified base and/or a modified phosphate backbone.

14. The method of claim 1 wherein the isolated nucleic acid is linear.

15. The method of claim 1 wherein the isolated nucleic acid is single stranded.

16. The method of claim 1 wherein the amount of isolated nucleic acid is about 0.01 to about 10.0 μg/ml.

17. The method of claim 1 wherein the majority of residues in the isolated nucleic are G and/or T residues.

18. The method of claim 1 wherein the sample is contacted with the isolated nucleic acid before the ligand comprising Cy5.

19. The method of claim 1 wherein the isolated nucleic acid is contacted with the ligand comprising Cy5 before the sample.

20. The method of claim 1 wherein the contacting occurs at room temperature.

21. The method of claim 1 wherein the Cy5 is Cy5.5.

22. The method of claim 1 further comprising contacting the sample with one or more fluorophore labeled ligands labeled with a fluorophore other than Cy5.

23. A method to reduce or eliminate nonspecific fluorescence by Cy5 labeled antibodies in a sample comprising cells suspected of expressing FcR, comprising: contacting the sample with a Cy5 labeled antibody and an amount of isolated nucleic acid effective to reduce or eliminate nonspecific fluorescence by the Cy5 labeled antibodies, thereby yielding a mixture.

24. The method of claim 23 further comprising contacting the sample with one or more fluorophore labeled antibodies labeled with a fluorophore other than Cy5.

25. The method of claim 22 or 24 further comprising detecting or determining Cy5 specific fluorescence of the mixture.

26. The method of claim 23 wherein the sample comprises monocytes.

27. The method of claim 23 wherein the sample comprises mammalian peripheral blood mononuclear cells.

28. The method of claim 27 wherein the cells are isolated human peripheral blood mononuclear cells.

29. The method of claim 23 wherein the cells express recombinant FcR.

30. The method of claim 23 wherein the CyS labeled antibodies further comprise PE.

31. The method of claim 23 wherein the Cy5 labeled antibodies are humanized antibodies.

32. The method of claim 23 wherein the antibodies are monoclonal antibodies.

33. The method of claim 23 wherein the isolated nucleic acid includes one or more oligonucleotides.

34. The method of claim 23 wherein the isolated nucleic has a phosphorothioate backbone.

35. The method of claim 23 wherein the isolated nucleic acid is isolated oligonucleotides each having the same sequence.

36. The method of claim 23 wherein the isolated nucleic acid include nucleotides with a modified sugar, a modified base and/or a modified phosphate backbone.

37. The method of claim 23 wherein the isolated nucleic acid is linear.

38. The method of claim 23 wherein the isolated nucleic acid molecule is single stranded.

39. The method of claim 23 wherein the amount of isolated nucleic acid is about 0.01 to about 10.0 μg/ml.

40. The method of claim 23 wherein the majority of residues in isolated nucleic are G and/or T residues.

41. The method of claim 23 wherein the sample is contacted with the isolated nucleic acid before the Cy5 labeled antibody.

42. The method of claim 23 wherein the isolated nucleic acid is contacted with the Cy5 labeled antibody before the sample.

43. The method of claim 23 wherein the contacting occurs at room temperature.

44. The method of claim 23 wherein the Cy5 is Cy5.5.

45. The method of claim 1 or 23 wherein the sample is a blood sample.

46. The method of claim 1, 23 or 24 further comprising subjecting the mixture to flow cytometry.

47. The method of claim 25 wherein fluorescence is detected or determined using a flow cytometer or a microscope.

48. The method of claim 24 wherein one antibody is labeled with fluorescein isothiocyanate (FITC) or PE.

49. The method of claim 22 wherein one ligand is labeled with fluorescein isothiocyanate (FITC) or PE.

50. A composition comprising a Cy5 labeled antibody and isolated nucleic acid in an amount effective to reduce or eliminate nonspecific fluorescence by Cy5.

51. The composition of claim 50 wherein the isolated nucleic acid includes one or more oligonucleotides.

52. The composition of claim 50 wherein the isolated nucleic acid has a phosphorothioate backbone.

53. The composition of claim 50 wherein the isolated nucleic acid is isolated oligonucleotides each having the same sequence.

54. The composition of claim 50 wherein the amount of isolated nucleic acid is about 0.01 to about 10.0 μg/ml.

55. A kit comprising:
a Cy5 labeled ligand;
isolated nucleic acid; and
instructions for reducing nonspecific fluorescence by Cy5 with the isolated nucleic acid.

56. The kit of claim 55 wherein the isolated nucleic acid is phosphorothioate oligonucleotides.

57. The kit of claim 55 wherein the isolated nucleic acid includes one or more oligonucleotides.

58. The kit of claim 55 wherein the isolated nucleic acid is isolated oligonucleotides each having the same sequence.

59. The kit of claim 55 wherein the isolated nucleic acid include nucleotides with a modified sugar, a modified base and/or a modified phosphate backbone.
